(19) United States
(12) Patent Application Publication
(10) Pub. No.: US 2006/0099714 A1
(43) Pub. Date: May 11, 2006

(54) Detection and analysis of ophthalmically-relevant fluorescent molecules

(75) Inventors: Nathan L. Mata, La Jolla, CA (US); Kenneth Widder, Rancho Santa Fe, CA (US); Jay Lichter, San Diego, CA (US)

Correspondence Address: WILSON SONSINI GOODRICH & ROSATI, 650 PAGE MILL ROAD, PALO ALTO, CA, 94304-1050, US

(73) Assignee: Sytera, Inc., La Jolla, CA (US)

(21) Appl. No.: 11/258,504

(22) Filed: Oct. 25, 2005

Related U.S. Application Data
(60) Provisional application No. 60/622,213, filed on Oct. 25, 2004.
(60) Provisional application No. 60/629,695, filed on Nov. 19, 2004.
(60) Provisional application No. 60/660,904, filed on Mar. 11, 2005.
(60) Provisional application No. 60/672,405, filed on Apr. 18, 2005.

(51) Int. Cl. G01N 33/50

(52) U.S. Cl. 436/106; 436/86

(57) ABSTRACT

Disclosed herein are methods and devices for detecting fluorescent molecules that are relevant to the health of the eye and related tissues. The presence of such molecules in the eye and related tissues can be used to diagnose whether the patient has certain diseases, including the macular degenerations and macular dystrophies. The amount of such molecules in the eye and related tissues can be used to determine the extent and stage of these diseases, to monitor the progress of these diseases, to design treatment strategies, to monitor the effectiveness of such treatments and to develop new therapies.

### CROSS-REFERENCE TO RELATED APPLICATIONS

This Application claims the benefit of U.S. Provisional Applications Ser. Nos. 60/622,213 filed Oct. 25, 2004, 60/629,695, filed on Nov. 19, 2004, 60/660,904, filed on Mar. 11, 2005, and 60/672,405, filed on Apr. 18, 2005, the disclosures of all of which are hereby incorporated by reference in their entirety.

### FIELD OF THE INVENTION

The methods and compositions described herein are directed to the treatment of ophthalmic conditions.

### BACKGROUND OF THE INVENTION

The early diagnosis of macular degenerations and/or macular dystrophies is important in order to initiate prompt therapeutic interventions. Macular degenerations include age-related macular degenerations (ARMD), which include wet and dry forms of ARMD. The dry form of ARMD, which accounts for about 90 percent of all cases, is also known as atrophic, nonexudative, or drusenoid macular degeneration. With the dry form of ARMD, drusen typically accumulate in the retinal pigment epithelium (RPE) tissue beneath/within the Bruch's membrane. Vision loss can then occur when drusen interfere with the function of photoreceptors in the macula. The dry form of ARMD results in the gradual loss of vision over many years. The dry form of ARMD can lead to the wet form of ARMD. The wet form of ARMD can progress rapidly and cause severe damage to central vision. The macular dystrophies include Stargardt Disease, also known as Stargardt Macular Dystrophy or Fundus Flavimaculatus, which is the most frequently encountered juvenile onset form of macular dystrophy.

### SUMMARY OF THE INVENTION

Presented herein are methods for detecting and/or measuring the presence of fluorescent compounds, including fluorescent compounds in ocular and/or ophthalmic samples. Also presented herein are methods for detecting and/or measuring fluorescent compounds in at least one eye of a mammal. Also presented herein are fluorescence-based diagnostic methods. Also presented herein are fluorescence-based analytical methods. Also presented herein are fluorescence-based methods that can be used as part of a therapy. Also presented herein are devices, instruments and/or tools for measuring fluorescent compounds. Also presented herein are treatment methods for ophthalmic conditions, including macular degnerations and dystrophies and geographic atrophy, comprising detecting and/or measuring the presence of fluorescent compounds, including fluorescent compounds in ocular and/or ophthalmic samples, and administration of a compound that reduces serum retinol levels.

Presented herein are methods for measuring the presence of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine in a sample. In one aspect, measuring the presence of these compounds is performed by illuminating the sample with light having specific wavelengths and measuring the emission fluorescence from the sample between other specified wavelengths. In other aspects, the methods are performed by using specific samples. In other aspects, the methods are performed by using received light from specific sources. In other aspects, the methods are performed by using various light sources. In other aspects, the methods use fluorescence to measure effectiveness, diagnose conditions, or monitor formation of drusen, lipofuscin, all-trans-retinal, and/or all-trans-retinal-lipid conjugates in the eye of a mammal. In other aspects, the methods are used to aid treatment of ophthalmic conditions by a variety of modalities.

In one aspect is a method for measuring the presence of N-retinylidene-phosphatidylethanolamine in a sample comprising illuminating the sample with light having a wavelength between 210 and 450 nm, and measuring the emission fluorescence from the sample between 470 and 700 nm.

In another aspect is a method for measuring the presence of dihydro-N-retinylidene-N-retinyl-phosphatidylethanolamine in a sample comprising illuminating the sample with light having a wavelength between 300 and 550 nm, and measuring the emission fluorescence from the sample between 570 and 700 nm.

In another aspect is a method for measuring the presence of N-retinylidene-N-retinyl-phosphatidylethanolamine in a sample comprising illuminating the sample with light having a wavelength between 300 and 550 nm, and measuring the emission fluorescence from the sample between 570 and 700 nm.

In another aspect is a method for measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm.

In another aspect is a method for measuring the presence of N-retinylidene-phosphatidylethanolamine in a sample comprising measuring the emission fluorescence from the sample between 510 and 700 nm, wherein the source light has a wavelength between 300 and 440 run.

In another aspect is a method for measuring the presence of dihydro-N-retinylidene-N-retinyl-phosphatidylethanolamine and/or N-retinylidene-N-retinyl-phosphatidylethanolamine in a sample comprising measuring the emission fluorescence from the sample between 570 and 700 nm, wherein the source light has a wavelength between 470 and 540 nm.

In another aspect is a method for measuring the presence of dihydro-N-retinylidene-N-retinyl-phosphatidylethanolamine and/or N-retinylidene-N-retinyl-phosphatidylethanolamine in a sample comprising measuring the emission fluorescence from the sample between 570 and 700 nm, wherein the source light has a wavelength between 480 and 530 nm.

In another aspect is a method for measuring the presence of dihydro-N-retinylidene-N-retinyl-phosphatidylethanolamine and/or N-retinylidene-N-retinyl-phosphatidylethanolamine in a sample comprising measuring the emission fluorescence from the sample between 570 and 700 nm, wherein the source light has a wavelength between 490 and 520 nm.

In another aspect is a method for measuring the presence of dihydro-N-retinylidene-N-retinyl-ethanolamine in a sample comprising measuring the emission fluorescence from the sample between 470 and 700 nm, and wherein the source light has a wavelength between 220 and 460 nm.

In another aspect is a method for measuring the presence of N-retinylidene-phosphatidylethanolamine and/or dihydro-N-retinylidene-N-retinyl-ethanolamine in a sample comprising illuminating the sample with light having a wavelength between 210 and 450 nm and/or 220 and 460 nm, and wherein the received light has a wavelength between 470 and 700 nm.

In another aspect is a method for measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and wherein the received light has a wavelength between 570 and 650 nm.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the preparation of the sample comprises dispersing at least one retina from an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a retina from an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a retinal pigment epithelium of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a Bruch's membrane of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a rod of an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a cone of an eye of a mammal.

In another aspect is a method for measuring the presence of dihydro-N-retinylidene-N-retinyl-phosphatidylethanolamine and/or N-retinylidene-N-retinyl-phosphatidylethanolamine in a sample comprising measuring the emission fluorescence from the sample between 500 and 700 nm, wherein the source light has a wavelength between 300 and 550 nm, wherein the eye has been removed from the mammal; alternatively, the method comprises measuring the emission fluorescence from the sample between 480 and 530 nm, wherein the source light has a wavelength between 300 and 550 nm, wherein the eye has been removed from the mammal.

In another aspect is a method for measuring the presence of dihydro-N-retinylidene-N-retinyl-phosphatidylethanolamine and/or N-retinylidene-N-retinyl-phosphatidylethanolamine in a sample comprising measuring the emission fluorescence from the sample between 500 and 700 nm, wherein the source light has a wavelength between 300 and 550 nm, andwherein the eye has not been removed from the mammal; alternatively the method comprises measuring the emission fluorescence from the sample between 480 and 530 nm, wherein the source light has a wavelength between 300 and 550 nm, andwherein the eye has not been removed from the mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, includes one or more of the following further limitations:
where the source light has a wavelength between 300 and 440 nm; between 470 and 540 nm; between 480 and 530 nm; between 490 and 520 nm; and between 415 and 445 nm;
wherein the received light has a wavelength between 450 and 550 nm; and between 550 and 650 nm;
wherein the preparation of the sample comprises dispersing at least one retina or retinal pigment epithelium eyecup from an eye of a mammal;
wherein the sample comprises a retina or retinal pigment epithelium eyecup from an eye of a mammal; and
wherein the sample comprises the eye of a rodent.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises an eye of a rodent and/or wherein the rodent is alive.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 run, includes one or more of the following further limitations:
where the source light has a wavelength between 300 and 440 nm; between 470 and 540 nm; between 480 and 530 nm; between 490 and 520 nm; or between 415 and 445 nm;
wherein the received light has a wavelength between 450 and 550 nm; or between 550 and 650;
wherein the preparation of the sample comprises dispersing at least one retina or retinal pigment epithelium eyecup from an eye of a mammal;
wherein the sample comprises a retina or retinal pigment epithelium eyecup from an eye of a mammal; and
wherein the sample comprises the eye of a primate.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a Bruch's membrane of a primate, and/or further wherein the primate is alive.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the sample comprises a rod of an eye of a primate and/or wherein the primate is a human.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 run, and measuring the emission fluorescence from the sample between 470 and 700 nm, are wherein the providing light step comprises filtering light through one or more narrow band pass filters.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises filtering light through a polarization filter or a bandpass filter or both.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a barrier filter.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 run, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 run, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of confocal microscopy.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of confocal microscopy or further wherein the confocal microscopy is confocal scanning ophthalmoscopy.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises magnification of an image.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of confocal microscopy and/or wherein the receiving light step comprises magnification of an image, or further wherein the magnification is from 2 to 500 times.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of confocal microscopy and/or wherein the receiving light step comprises magnification of an image, or further wherein the magnification is from 2 to 500 times, and even further wherein the magnification is from 10 to 100 times.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a photodetector.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 run, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a microprocessor.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 run, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a photodiode and/or photodiode array.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a photomultiplier tube.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a camera and/or a video camera.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises use of a charge-coupled device.

Further embodiments of the method for (a) measuring the presence of N-retinylidene-phosphatidylethanolamine in a sample comprising illuminating the sample with light having a wavelength between 210 and 450 m, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises storage of an image.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises spatial determination of the received light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the receiving light step comprises constructing a computer image of the positions and values of the received light in the sample.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of a microprocessor.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of non-polarized light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of polarized light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of a filament lamp source.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of a discharge lamp source.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 run, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of a laser.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of a light-emitting diode.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of pulsed light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of constant light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of coherent light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 run, are wherein the providing light step comprises use of non-coherent light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 run, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the providing light step comprises use of whole eye illumination.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising measuring the fluorescence of light from the sample between 700 and 900 nm.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 run, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising measuring the fluorescence of light from the sample between 700 and 900 nm and further comprising determining the ratio of absorbance measured between 360 to 460 nm and the fluorescence measured between 700 and 900 nm.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising measuring the fluorescence of light from the sample between 700 and 900 nm and/or further comprising determining the ratio of absorbance measured between 450 to 550 nm and the fluorescence measured between 700 and 900 nm.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 run, and measuring the emission fluorescence from the sample between 470 and 700 nm, further comprising measuring the fluorescence of light from the sample between 700 and 900 nm and further comprising determining the ratio of absorbance measured between 475 to 525 nm and the fluorescence measured between 700 and 900 nm.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, and (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising measuring the fluorescence of light from the sample between 700 and 900 nm and further comprising determining the ratio of absorbance measured between 575 to 650 nm and the fluorescence measured between 700 and 900 nm.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising use of spatial filtering to reduce non-specific background light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to measure the effectiveness of a drug candidate.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to measure the effectiveness of a treatment.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to diagnose an ophthalmic disease or condition.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 run, and measuring the emission fluorescence from the sample between 470 and 700 nm, are wherein the fluorescence is used to diagnose an ophthalmic disease or condition, including wherein the ophthalmic disease or condition is Stargardt Disease.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to diagnose an ophthalmic disease or condition, including wherein the ophthalmic disease or condition is dry form age-related macular degeneration.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to diagnose an ophthalmic disease or condition, including wherein the ophthalmic disease or condition is wet form age-related macular degeneration.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to monitor or as a surrogate measurement for the formation of drusen in an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to monitor or as a surrogate measurement for the formation of lipofuscin in an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to monitor or as a surrogate measurement for the formation of all-trans-retinal in an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor for an ophthalmic disease or condition in an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor for an ophthalmic disease or condition in an eye of a mammal, including wherein the ophthalmic disease or condition is Stargardt Disease, and/or dry form age-related macular degeneration.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to measure the effectiveness of a treatment, including wherein the treatment comprises administration of an all-trans retinyl derivative and/or administration of a 13-cis-retinyl derivative, and/or administration of a vitamin, and/or administration of an anti-oxidant, and/or administration of a mineral, and/or administration of a nitric oxide inducer, and/or negatively-charged phospholipids, and/or anti-inflammatory agent, and/or rheophoresis, and/or laser photocoagulation.

In further embodiments comprising administration of an all-trans retinyl derivative, the all-trans retinyl derivative is administered at least once in an effective amount and has the structure of Formula (I): 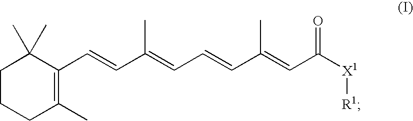
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)— NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle, provided that R³ is not H when both x is 0 and L¹ is a single bond; or an active metabolite, or a pharmaceutically acceptable prodrug or solvate thereof.

In further embodiments (a) X¹ is NR², wherein R² is H or (C₁-C₄)alkyl; (b) wherein x is 0; (c) x is 1 and L¹ is —C(O)—; (d) R³ is an optionally substituted aryl; (e) R³ is an optionally substituted heteroaryl; (f) X¹ is NH and R³ is an optionally substituted aryl, including yet further embodiments in which (i) the aryl group has one substituent, (ii) the aryl group has one substituent selected from the group consisting of halogen, OH, O(C₁-C₄)alkyl, NH(C₁-C₄)alkyl, O(C₁-C₄)fluoroalkyl, and N[(C₁-C₄)alkyl]₂, (iii) the aryl group has one substituent, which is OH, (v) the aryl is a phenyl, or (vi) the aryl is naphthyl; (g) the compound is 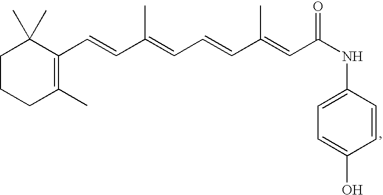
or an active metabolite, or a pharmaceutically acceptable prodrug or solvate thereof; (h) the compound is 4-hydroxyphenylretinamide, or a metabolite, or a pharmaceutically acceptable prodrug or solvate thereof; (i) the compound is 4-methoxyphenylretinamide, or (j) 4-oxo fenretinide, or a metabolite, or a pharmaceutically acceptable prodrug or solvate thereof.

In further embodiments, the administration of a compound of Formula (I) is used to treat ophthalmic conditions by (a) lowering the levels of serum retinol in the body of a patient.

In further embodiments (a) the effective amount of the compound is systemically administered to the mammal; (b) the effective amount of the compound is administered orally to the mammal; (c) the effective amount of the compound is intravenously administered to the mammal; (d) the effective amount of the compound is ophthalmically administered to the mammal; (e) the effective amount of the compound is administered by iontophoresis; or (f) the effective amount of the compound is administered by injection to the mammal.

In further embodiments the mammal is a human, including embodiments wherein (a) the human is a carrier of the mutant ABCA4 gene for Stargardt Disease or the human has a mutant ELOV4 gene for Stargardt Disease, or has a genetic variation in complement factor H associated with age-related macular degeneration, or (b) the human has an ophthalmic condition or trait selected from the group consisting of Stargardt Disease, recessive retinitis pigmentosa, geographic atrophy (of which scotoma is one non-limiting example), photoreceptor degeneration, dry-form AMD, recessive cone-rod dystrophy, exudative age-related macular degeneration, cone-rod dystrophy, and retinitis pigmentosa. In further embodiments the mammal is an animal model for retinal degeneration.

In further embodiments, are methods comprising multiple administrations of the effective amount of the compound, including further embodiments in which (i) the time between multiple administrations is at least one week; (ii) the time between multiple administrations is at least one day; and (iii) the compound is administered to the mammal on a daily basis; or (iv) the compound is administered to the mammal every 12 hours. In further or alternative embodiments, the method comprises a drug holiday, wherein the administration of the compound is temporarily suspended or the dose of the compound being administered is temporarily reduced; at the end of the drug holiday, dosing of the compound is resumed. The length of the drug holiday can vary from 2 days to 1 year.

In further embodiments are methods comprising administering at least one additional agent selected from the group consisting of an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, 13-cis-retinoic acid (including derivatives of 13-cis-retinoic acid), 11-cis-retinoic acid (including derivatives of 11-cis-retinoic acid), 9-cis-retinoic acid (including derivatives of 9-cis-retinoic acid), and retinylamine derivatives. In further embodiments:
(a) the additional agent is an inducer of nitric oxide production, including embodiments in which the inducer of nitric oxide production is selected from the group consisting of citrulline, ornithine, nitrosated L-arginine, nitrosylated L-arginine, nitrosated N-hydroxy-L-arginine, nitrosylated N-hydroxy-L-arginine, nitrosated L-homoarginine and nitrosylated L-homoarginine;
(b) the additional agent is an anti-inflammatory agent, including embodiments in which the anti-inflammatory agent is selected from the group consisting of a non-steroidal anti-inflammatory drug, a lipoxygenase inhibitor, prednisone, dexamethasone, and a cyclooxygenase inhibitor;
(c) the additional agent is at least one physiologically acceptable antioxidant, including embodiments in which the physiologically acceptable antioxidant is selected from the group consisting of Vitamin C, Vitamin E, beta-carotene, Coenzyme Q, and 4-hydroxy-2,2,6,6-tetramethylpiperadine-N-oxyl, or embodiments in which (i) the at least one physiologically acceptable antioxidant is administered with the compound having the structure of Formula (I), or (ii) at least two physiologically acceptable antioxidants are administered with the compound having the structure of Formula (I);
(d) the additional agent is at least one physiologically acceptable mineral, including embodiments in which the physiologically acceptable mineral is selected from the group consisting of a zinc (II) compound, a Cu(II) compound, and a selenium (II) compound, or embodiments further comprising administering to the mammal at least one physiologically acceptable antioxidant;
(e) the additional agent is a negatively charged phospholipid, including embodiments in which the negatively charged phospholipid is phosphatidylglycerol;
(f) the additional agent is a carotenoid, including embodiments in which the carotenoid is selected from the group consisting of lutein and zeaxanthin;
(g) the additional agent is a statin, including embodiments in which the statin is selected from the group consisting of rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium, and dihydrocompactin;
(h) the additional agent is an anti-angiogenic drug, including embodiments in which the the anti-angiogenic drug is Rhufab V2, Tryptophanyl-tRNA synthetase, an Anti-VEGF pegylated aptamer, Squalamine, anecortave acetate, Combretastatin A4 Prodrug, Macugen™, mifepristone, subtenon triamcinolone acetonide, intravitreal crystalline triamcinolone acetonide, AG3340, fluocinolone acetonide, and VEGF-Trap;
(i) the additional agent is a matrix metalloproteinase inhibitor, including embodiments in which the matrix metalloproteinase inhibitor is a tissue inhibitors of metalloproteinases, α₂-macroglobulin, a tetracycline, a hydroxamate, a chelator, a synthetic MMP fragment, a succinyl mercaptopurine, a phosphonamidate, and a hydroxaminic acid;
(j) the additional agent is 13-cis-retinoic acid (including derivatives of 13-cis-retinoic acid), 11-cis-retinoic acid (including derivatives of 11-cis-retinoic acid), or 9-cis-retinoic acid (including derivatives of 9-cis-retinoic acid);
(k) the additional agent is a retinylamine derivative, including an all-trans-retinylamine derivative, a 13-cis-retinylamine derivative, a 11-cis-retinylamine derivative, or a 9-cis-retinylamine derivative;
(l) the additional agent is administered (i) prior to the administration of the compound having the structure of Formula (I), (ii) subsequent to the administration of the compound having the structure of Formula (I), (iii) simultaneously with the administration of the compound having the structure of Formula (I), or (iv) both prior and subsequent to the administration of the compound having the structure of Formula (I); or
(m) the additional agent and the compound having the structure of Formula (I), are administered in the same pharmaceutical composition.

In further embodiments are methods comprising administering extracorporeal rheopheresis to the mammal.

In further embodiments are methods comprising administering to the mammal a therapy selected from the group consisting of limited retinal translocation, photodynamic therapy, drusen lasering, macular hole surgery, macular translocation surgery, Phi-Motion, Proton Beam Therapy, Retinal Detachment and Vitreous Surgery, Scleral Buckle, Submacular Surgery, Transpupillary Thermotherapy, Photosystem I therapy, MicroCurrent Stimulation, anti-inflammatory agents, RNA interference, administration of eye medications such as phospholine iodide or echothiophate or carbonic anhydrase inhibitors, microchip implantation, stem cell therapy, gene replacement therapy, ribozyme gene therapy, photoreceptor/retinal cells transplantation, and acupuncture.

In further embodiments are methods comprising the use of laser photocoagulation to remove drusen from the eye of the mammal.

In further embodiments are methods comprising administering to the mammal at least once an effective amount of a second compound having the structure of Formula (I), wherein the first compound is different from the second compound.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a surrogate marker for an ophthalmic disease or condition in an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 run, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor for an ophthalmic disease or condition in an eye of a mammal, wherein the ophthalmic disease or condition results from a mutation in the ABCA4 gene relative to wild type.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor for an ophthalmic disease or condition in an eye of a mammal, wherein the ophthalmic disease or condition is cone-rod dystrophy.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor for an ophthalmic disease or condition in an eye of a mammal, wherein the ophthalmic disease or condition is Stargardt Disease.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor for an ophthalmic disease or condition in an eye of a mammal, wherein the ophthalmic disease or condition is dry form age-related macular degeneration.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor or functional marker for an ophthalmic disease or condition in an eye of a mammal, wherein the ophthalmic disease or condition comprises accumulation of drusen.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor or functional marker for an ophthalmic disease or condition in an eye of a mammal, wherein the ophthalmic disease or condition comprises disruption of the visual cycle.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor or functional marker for an ophthalmic disease or condition in an eye of a mammal, wherein the ophthalmic disease or condition comprises accumulation of lipofuscin.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used as a risk factor or functional marker for an ophthalmic disease or condition in an eye of a mammal, wherein the ophthalmic disease or condition comprises sensitivity to light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to monitor the progress of an ophthalmic disease or condition in an eye of a mammal.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to determine whether the visual cycle of an eye of a mammal has been disrupted.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to determine whether an eye of a mammal should be protected from light.

Further embodiments of the method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, are wherein the fluorescence is used to determine whether an eye of a mammal should receive treatment for an ophthalmic disease or condition.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising designing a treatment for an eye of a mammal.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising designing a treatment for an eye of a mammal, wherein the eye of the mammal is at risk for developing an ophthalmic disease or condition.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising designing a treatment for an eye of a mammal, wherein the eye of the mammal has an ophthalmic disease or condition.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising testing a mammal for an ophthalmic disease or condition.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising testing a mammal for an ophthalmic disease or condition, wherein the testing comprises genetic testing.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising repeating the providing light step and the receiving light step.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising repeating the providing light step and the receiving light step, wherein the time between each repetition is less than 5 minutes.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising repeating the providing light step and the receiving light step, wherein the time between each repetition is more than 5 minutes.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and. 700 nm, further comprising repeating the providing light step and the receiving light step, wherein the time between each repetition is more than 1 hour.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising repeating the providing light step and the receiving light step, wherein the time between each repetition is more than 1 day.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 run, and measuring the emission fluorescence from the sample between 470 and 700 nm, further comprising repeating the providing light step and the receiving light step, wherein the time between each repetition is more than 1 week.

Further embodiments are a method for (a) measuring the presence of N-retinylidene-phosphatidylethanolamine in a sample comprising illuminating the sample with light having a wavelength between 210 and 450 nm, and measuring the emission fluorescence from the sample between 470 and 700 nm, (b) measuring the presence of dihydro-N-retinylidene-N-retinyl-phosphatidylethanolamine and/or N-retinylidene-N-retinyl-phosphatidylethanolamine in a sample comprising illuminating the sample with light having a wavelength between 300 and 550 nm, and measuring the emission fluorescence from the sample between 570 and 700 nm, and/or (c) measuring the presence of dihydro-N-retinylidene-N-retinyl-ethanolamine in a sample comprising illuminating the sample with light having a wavelength between 220 and 460 nm, and measuring the emission fluorescence from the sample between 470 and 700 nm, further comprising deconvoluting the received light from the sample.

Other objects, features and advantages of the methods and compositions described herein will become apparent from the detailed description herein. It should be understood, however, that the detailed description and the specific examples, while indicating specific embodiments, are given by way of illustration only, since various changes and modifications within the spirit and scope of the invention will become apparent to those skilled in the art from this detailed description.

All references cited herein, including patents, patent applications, and publications, are hereby incorporated by reference in their entirety.

### BRIEF DESCRIPTION OF THE FIGURES

FIG. 1 presents a schematic of methods for detecting and/or measuring the presence of fluorescent compounds in a sample.

FIG. 2 presents a schematic of devices for detecting and/or measuring the presence of fluorescent compounds in a sample.

FIG. 3 illustrates the anatomical organization of the vertebrate eye.

FIG. 4 illustrates the apical processes and outer segments of the retinal pigment epithelial cells.

FIG. 5 illustrates A2PE-H₂ absorption spectra and age-dependent accumulation in ABCA4^{-/-} ROS and retinal pigment epithelial cells.

FIG. 6 illustrates the biogenesis of A2E and A2E-oxiranes.

FIG. 7 illustrates A2PE-H₂ and A2E absorption spectra in normal and Stargardt's retinal pigment epithelial cells.

FIG. 8A illustrates a solid phase sample mount; FIG. 8B illustrates a modified sample cell carriage for live mice.

FIG. 9 illustrates absorbance and fluorescence spectra of A2E and A2PE-H₂ in an extract from ABCA4^{-/-} mouse eyecups.

FIG. 10 illustrates fluorescence emission spectra from an ABCA4^{-/-} retinal pigment epithelium/eyecup and retina: (A) data were acquired from samples which were separately flat-mounted on the solid phase sample mount, or (B) emission spectra were obtained from flat-mounted ABCA4^{-/-} retina explants that show age-dependent accumulation of a unique fluorophore.

FIG. 11A illustrates HPLC separation and absorption spectra of A2E, A2PE, and A2PE-H₂ fluorophores obtained from an ABCA4^{-/-} whole eyecup extract; FIG. 11B illustrates excitation and emission spectra obtained from HPLC purified A2E, A2PE, and A2PE-H₂.

### DETAILED DESCRIPTION OF THE INVENTION

Described herein are methods and devices for detecting and/or measuring certain fluorescent molecules that are relevant to the health of the eye and related tissues. Such molecules include, but are not limited to N-retinylidene-phosphatidylethanolamine, dihydro-N-retinylidene-N-retinyl-phosphatidylethanolamine, N-retinylidene-N-retinyl-phosphatidylethanolamine and/or dihydro-N-retinylidene-N-retinyl-ethanolamine. The information obtained from detecting and measuring such fluorescent compounds in the eye and/or related tissues can be used for diagnostic, therapeutic and/or analytical purposes.

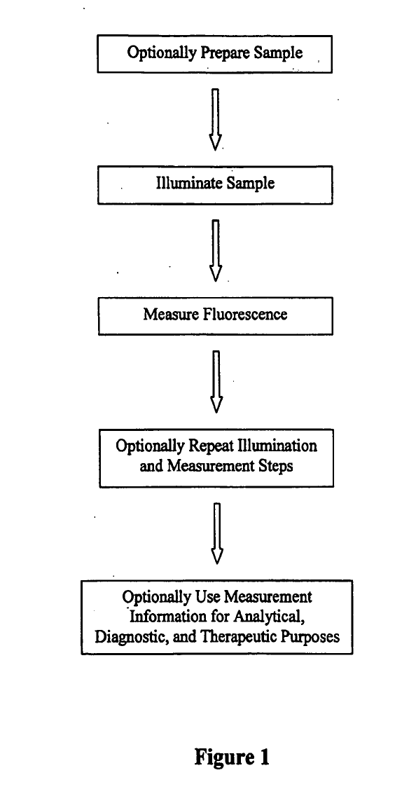

One of the methods for detecting and/or measuring fluorescent molecules is presented schematically in FIG. 1. An optional first step is the preparation of the sample. The sample can comprise either an eye or related tissues, a portion of an eye, or material derived from or extracted from an eye. The eye may be either in a living or dead animal or removed from an animal. Further, the methods and devices described herein may be used with a human patient, in which case, the sample is at least one of the eyes in the human patient. By way of example only, the eyes of a human patient may be examined using the methods and devices described herein as part of a routine ophthalmic examination. The human patient may be optionally anesthetized, but the methods and devices described herein do not require such treatment. Further, the methods and devices described herein may be used with test and/or laboratory animals, such as mice, rats, non-human primates, and the like. Such test and/or laboratory animals may be alive or dead. Further, the eyes may be removed from the test and/or laboratory animals and subsequently analyzed using the methods and devices described herein. Further, the eye may be dissected and portions of the eye studied separately. By way of example only, any one of the following tissues may be studied singly or in combination with any other tissue: the retina, the retinal pigment epithelium, the Bruch's membrane, at least one rod, and at least one cone. Further, the eye tissue, upon removal from the animal may be further prepared for analysis. Such further preparations include homogenization of the eye, dispersal or suspension in another media, and the like. Further, the samples may include or be derived from cultured cells or tissues, or from tissue banks, or from storage centers. In other words, the samples used in the methods and devices described herein originate from the eye of an animal, but are in no way limited by the methods (if any) for subsequently preparing the sample for analysis.

After optionally preparing the sample, the sample is illuminated with light. The methods and devices described herein are not limited by the type or source of light, that is, the light may originate, by way of example only, from a lamp, laser, or light-emitting diode. The light may be pulsed (in any sequence) or continuous; further the light may be coherent or non-coherent; further the light may be polarized or non-polarized; further the light may pass through filters (including, but not limited to band-pass filters), blocking (e.g., spatial filtering) and/or focusing devices; further the light may illuminate all (e.g., whole eye illumination) or only a portion of the sample. The wavelength range or ranges used for illuminating the sample depend upon the fluorescent compound or compounds to be detected, further detail is provided herein for specific compounds. Preferably, the wavelength(s) of light used in the illuminating step should excite the fluorescent compound so as to emit a fluorescence signal that can be subsequently detected and/or measured. In addition, the wavelength range used for illumination may also include wavelengths that are not well absorbed (if at all) by the fluorescence compounds of interest; such light may be used as a reference signal or for background subtraction: by way of example only, light in the range 700-900 nm is not well absorbed (if at all) by most fluorescent compounds that are components of (or derived therefrom) the visual cycle. If the purpose is to study such components or their derivatives, then the illuminating light can include light in the range 700-900 nm as a reference or background signal. However, the use of such a reference or background signal is not required by the methods and devices described herein. In addition, the absorbance of the illuminating light may also be measured and used separately or in combination with the fluorescence signal measured and/or detected in the detection step. Such an absorbance signal may be diagnostic for a particular fluorescent compound, as described elsewhere herein. The key requirement for the illumination step is that the fluorescent compound or compounds to be detected absorb at least a portion of the light applied to the sample. The use of a microprocessor may also be used to control the illumination step. In any sample, there may be more than one type of fluorescent compound; if more than one type of fluorescent compound is presented, then the illuminating light may be provided so as to be absorbed by only one type of compound or by multiple types of compounds.

After illuminating the sample, the fluorescence emitted from the fluorescent compounds in the sample is detected. Such emitted fluorescence may be detected by any number of methods, or a combination of methods. For example, the fluorescence signal may be detected at only one wavelength, at different wavelengths, at a range of wavelengths, or over multiple ranges of wavelengths. If a specific fluorescence is being detected and/or measured, then one of the methods described herein examines a specific range of wavelengths that corresponds to the major component

The information or data or images acquired from the detection step may be stored temporarily or permanently in a variety of media, including by way of example only, film, computer memory or any other form of archival material. Such record-keeping and/or storage of information, data and/or images is generally associated with patient diagnosis and treatment, as well as for testing the effectiveness of a drug or treatment (in vivo or in vitro). By storing or archiving such information, one of skill in the art can also create a temporal analysis of the sample. Furthermore, the archived information, data or images can be further processed (e.g., magnified, enriched, deconvoluted, pseudocolored, quantitated) as desired.

The optional sample preparation, the illumination of the sample, the detection of fluorescence and the optional storage of information can be considered one detection cycle. As such, it is contemplated herein the repetition of this detection cycle on a sample. Certainly, if the sample is already prepared, it may not be necessary to re-prepare the sample, especially if the time interval between detection cycles is short (e.g., less than 5 minutes or less than one hour or even less than one day). By way of example only, it may be necessary to repeat a detection cycle to ensure the accuracy of the measurements, in which case, the time interval between detection cycles may be relatively short. If the interval between detection cycles is longer, it may be necessary to store the sample (e.g., if in a non-living animal), continue with care of the laboratory animal if the sample is the eye of a laboratory animal, or request the human patient return for further studies if the sample is the eye of a human patient. In addition, if the sample is being provided with therapy or otherwise studied (e.g., in the testing and/or design of a new drug, or the treatment of a human patient with an ophthalmic condition), the interval between detection cycles may be used to provide further therapy or manipulation to the sample. The time between detection cycles may be less than 5 minutes, more than 5 minutes, more than one hour, more than one day, more than one week, and even more than one month. If the sample is the eye of a laboratory animal or human patient, it may be necessary to repeat the detection cycle at appropriate intervals throughout the life of the patient. The duration of time between detection cycles and the number of times the detection cycle is repeated is within the discretion of one of skill in the art. In any case, the duration of time between detection cycles does not have to be uniform and may be a combination of multiple repeat cycles; thus by way of illustration only, if the sample is in the eye of human patient, the detection cycle may be repeated every 2 minutes for a total of 10 times, and this mini-cycle then repeated at least once a month or at least once every 6 months for the life of the human patient.

The information collected from a detection cycle or cycles may be optionally used for a number of purposes in which the absorbance and/or fluorescence detected from a sample is used as a surrogate marker and/or risk factor for the status of a sample. Non-limiting examples include (a) measuring the effectiveness of a drug candidate for a relevant ophthalmic disease or condition (including the retinal and/or macular degenerations or dystrophies) in an in vitro sample or an in vivo sample (including the eye of a living laboratory animal, including an ABCA4 knockout mouse, or human patient) by measuring changes in the amount of fluorescent compound(s) in a sample following administration of the drug candidate to the sample; (b) measuring the effectiveness of a treatment for a relevant ophthalmic disease or condition (including the retinal and/or macular degenerations or dystrophies) in an in vivo sample (including the eye of a living laboratory animal, including an ABCA4 knockout mouse, or human patient) by measuring changes in the amount of fluorescent compound(s) in a sample following administration of a treatment to the laboratory animal or human patient; (c) diagnose or otherwise determine the risk of an ophthalmic disease or condition (including the retinal and/or macular degenerations, such as dry form and wet form age-related macular degeneration, or dystrophies, such as Stargardt Disease) in a human patient based on the presence and/or amount of fluorescent compound(s) detected in the eye of the human patient; (d) monitoring the formation of drusen and/or lipofuscin in the eye of a mammal (including a laboratory animal or a human patient) by measuring the presence and/or amount of fluorescent compound(s) detected in the eye of the mammal; (e) determine whether the visual cycle of the eye of a mammal has been disrupted by measuring the presence and/or amount of fluorescent compound(s) detected in the eye of the mammal; (f) monitor the progress of an ophthalmic disease or condition (including the retinal and/or macular degenerations, such as dry form and wet form age-related macular degeneration, or dystrophies, such as Stargardt Disease) in the eye of a mammal by measuring changes over time in the amount of fluorescent compound(s) in the eye of the mammal; and (g) determining whether a mammal should receive treatment for an ophthalmic disease or condition (including the retinal and/or macular degenerations, such as dry form and wet form age-related macular degeneration, or dystrophies, such as Stargardt Disease), and what that treatment should be, by measuring and/or monitoring the presence of fluorescent compound(s) in the eye of the mammal.

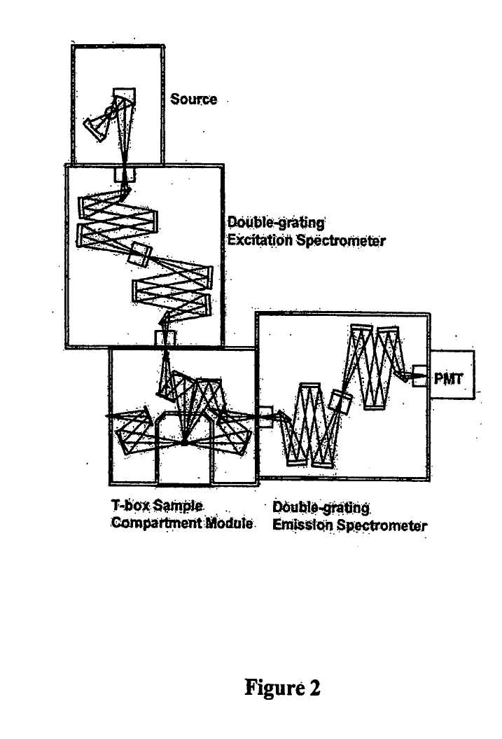

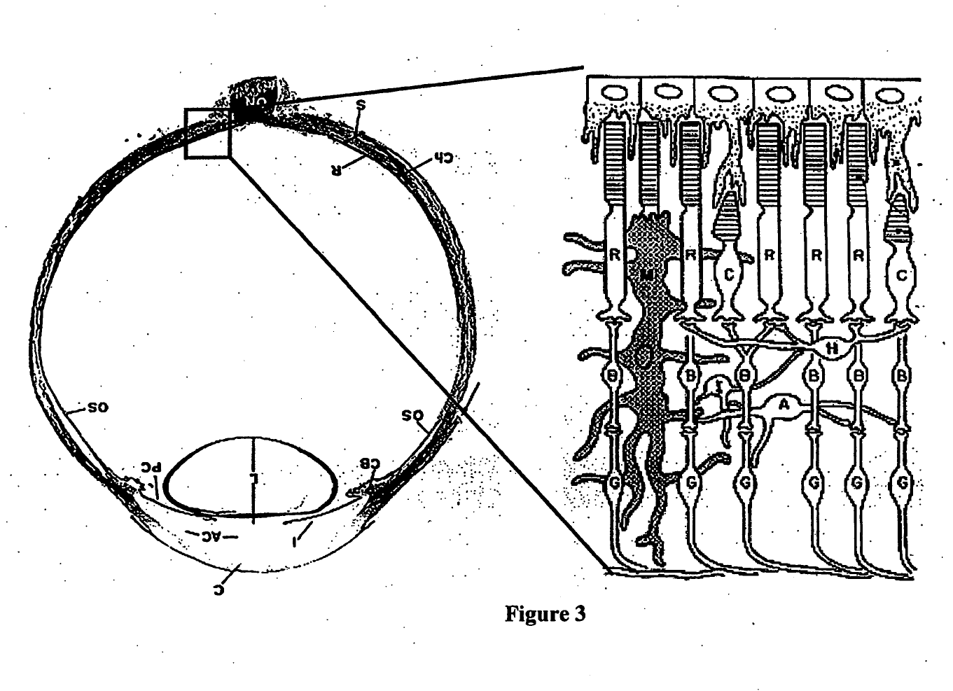

A schematic of one example of a device that may be used with the methods described herein is presented in FIG. 2; the various mirrors and lenses depicted within this figure are for illustrative purposes and not to provide a limitation to the design of the device that may be used with the detection, measurement and analytical methods described herein. The light is provided from a source (as described elsewhere herein) which is subsequently passed through a double-grating excitation spectrometer, which can comprise a series of mirrors and lenses. In addition the double-grating excitation spectrometer may include a microprocessor and associated software for controlling the action of the mirrors and lenses, as well as for recording any information regarding the properties of the light passing through the double-grating excitation spectrometer. Other methods and designs for manipulating, controlling and/or measuring the light prior to contact with the sample may be used in such a device.

After passing through the double-grating excitation spectrometer, the light passes through a sample compartment; in the case of FIG. 2, the sample compartment is designed as a T-box sample compartment module although other designs are considered well within the scope of the devices described herein. A series of lenses and mirrors may also be arranged within the sample module. In addition, the sample module may also reside within the double-grating spectrometers; i.e., the sample compartment does not have to exist as a distinct module. The components and properties of the sample compartment module may also be controlled, monitored and/or recorded using a microprocessor and associated software, or by means of an analog device, or more directly by the end-user of the device. After the source light interacts with the sample, the resultant light from the sample (via reflection, emission, transmission, and the like) can be further analyzed. A portion of the source light may also be used as a reference beam, in which case the reference beam may not make contact with the sample. In the example device presented schematically in FIG. 2, the resultant light (also described herein as the measured light and the received light) can further pass through a series of mirrors and lenses within the sample compartment; in addition, a portion of the resultant light may also be sent to other devices or instruments.

In FIG. 2, after passing through the series of optional mirrors and lenses in the sample compartment, the resultant light passes through a double-grating emission spectrometer, which may include a further series of lenses and mirrors. As with the double-grating excitation spectrometer, the double-grating emission spectrometer may include a microprocessor and associated software for controlling the action of the mirrors and lenses, as well as for recording any information regarding the properties of the light passing through the double-grating emission spectrometer. Other methods and designs for manipulating, controlling and/or measuring the light after contact with the sample may be used in such a device. In the final stage of the device presented schematically in FIG. 2, the resultant light interacts with a photo-multiplier tube, which can be used as part of an instrument for recording the properties of the resultant light. Methods for recording, storing and analyzing the properties of the resultant light are described herein and may be incorporated into the device presented schematically in FIG. 2. Such a device may also include a means for providing a series of measurements, including but not limited to, various timing devices, choppers, and associated hardware, microprocessors, data storage devices, and software.

Devices suitable for the methods describe herein may include software for controlling the illumination step, the detecting step, archiving information, manipulating or deconvoluting images, data or information from the detection step, and the like. By way of example only, confocal microscopes, including confocal scanning ophthalmoscopes, can be modified and used with the methods described herein. Such a device can be built from component parts or by modifying existing equipment. Such a device may exist as a series of modules or as a distinct, full-housed unit.

The Visual Cycle. The methods and devices described herein concern the visual cycle (cycle for regenerating rhodopsin), including methods and devices for monitoring, detecting or studying components of that pathway. The vertebrate retina contains two types of photoreceptor cells. Rods are specialized for vision under low light conditions. Cones are less sensitive, provide vision at high temporal and spatial resolutions, and afford color perception. Under daylight conditions, the rod response is saturated and vision is mediated entirely by cones. Both cell types contain a structure called the outer segment comprising a stack of membranous discs. The reactions of visual transduction take place on the surfaces of these discs. The first step in vision is absorption of a photon by an opsin-pigment molecule, which involves 11-cis to all-trans isomerization of the retinal chromophore. Before light sensitivity can be regained, the resulting all-trans-retinal must dissociate from the opsin apoprotein and isomerize to 11-cis-retinal.

All-trans-retinal is a visual cycle retinoid which upon condensation with phosphatidylethanolamine produces the diretinal species N-retinylidene-N-retinylethanolamine. 11-cis-retinal is the photoreactive portion of rhodopsin, which is converted to all-trans-retinal when a photon of light in the active absorption band strikes the molecule. This process goes through a sequence of chemical reactions as 11-cis-retinal isomerizes to all-trans-retinal. During this series of chemical steps, the nerve fiber, which is attached to that particular rod or cone, undergoes a stimulus that is perceived in the brain as a visual signal.

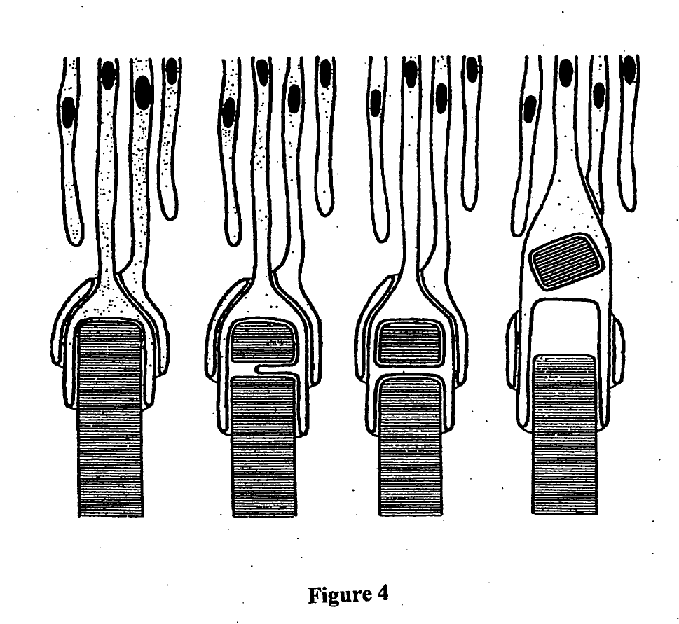

Anatomical Organization of the Vertebrate Eye. With reference to FIG. 3, the eye is a complex organ composed of many parts. Good vision depends on the way in which those parts work together. As light enters the eye, it passes through the cornea C, lens L, vitreous filled center, then finally falls upon the retina. The retina is a thin, light-sensitive tissue lining the back of the eye. The retina converts light patterns into information the brain can use. The macula is the small central portion of the retina with the most dense population of photoreceptors, the light sensing cells. The retina is composed of many different tissue layers, each with a specific function. The cross-section in FIG. 3 shows an enlarged view of the retina. The photoreceptor layer is composed of light-sensitive cells called rods R and cones C. Light images are converted into electrochemical signals inside the photoreceptors. On top of the photoreceptors is a dark layer called the retinal pigment epithelium or RPE, see FIGS. 3 and 4 cross-section. Cells of the RPE absorb excess light and transport oxygen, nutrients and cellular wastes between the photoreceptors and the choroid. Bruch's membrane separates the blood vessels of the choroid from the RPE layer, see FIG. 3. The choroid Ch is a layer of blood vessels that supplies oxygen and nutrients to the outer layers of the retina. The scleara S, is the fibrous, white, outer covering of the eye.

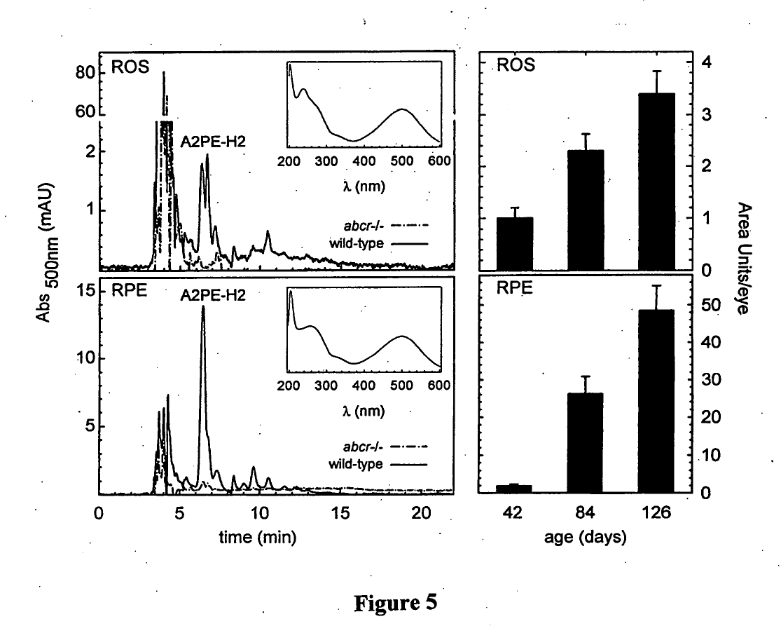

Visual Cycle for Regeneration of Rhodopsin. Rhodopsin, G protein-coupled receptor, has two physiological pathways: phototransduction/recovery from bleaching (return of activated components to the dark state) and the retinoid cycle (production of 11-cis-retinal), FIG. 5. Vertebrate phototransduction is initiated by a photochemical reaction whereby 11-cis-retinal bound to its opsin moiety (rhodopsin=opsin+11-cis-retinal) undergoes isomerization to all-trans-retinal producin conformation changes in opsin. In vertebrates, restoration of a photosensitive receptor conformation (return to the dark state) requires the formation of 11-cis-retinal from all-trans-retinal via the retinoid cycle. The entire cycle of isomerization and pigment regeneration in humans occurs on a time scale of minutes for rhodopsin, and significantly faster for cone pigments. Reduction of all trans-retinal to all-trans-retinol takes place in photoreceptor outer segments whereas all other reactions, including isomerization, occur within retinal pigment epithelials cells (RPE). The all-trans-retinylidene Schiff base hydrolyzes and all-trans-retinal dissociates from the binding pocket of opsin, yet the molecular steps leading to its release from the opsin-binding pocket remain not fully explained. Removal of all-trans-retinal from the disks may be facilitated by an ATP-binding cassette transporter (ABCA4), mutations in which are causative of an array of retina disease including Stargardt's Disease, cone-rod dystrophy, retinitis pigmentosa and possibly macular degeneration.

Further, all-trans-retinal is reduced to all-trans-retinol by NADPH-dependent all-trans-retinol dehydrogenase, a membrane-associated enzyme that belongs to large gene family of short-chain alcohol dehydrogenases (SCAD). All-trans-retinol translocates to the RPE via a poorly defined process, perhaps involving components like IRBP and RBP present in the interphotoreceptor matrix (IPM), or passive diffusion driven by trapping retinoids (e.g., insoluble fatty acid retinyl esters) in RPE. Esterification in the RPE involves the transfer of an acyl group from lecithin to retinol and is catalyzed by lecithin:retinol acyltransferase (LRAT). These esters may be substrates for an as yet unknown enzyme termed isomerohydrolase, which would use the energy of retinyl ester hydrolysis to isomerase all-trans-retinol to 11-cis-retinol and thus, drive the reaction forward. Alternatively, these two reactions may proceed separately, i.e., the ester may be first hydrolyzed by a retinyl ester hydrolase and then isomerized to 11-cis-retinol through an intermediate. 11-cis-retinol would then be oxidized to 11-cis-retinal in a reaction catalyzed by NAD- and NADP-dependent 11-cis-retinol dehydrogenases, which are other short chain dehydrogenase family members. Finally 11-cis-retinal moves back to the rod photoreceptors, either in IRBP-dependent or -independent fashion, where it joins with opsin to regenerate visual pigment.

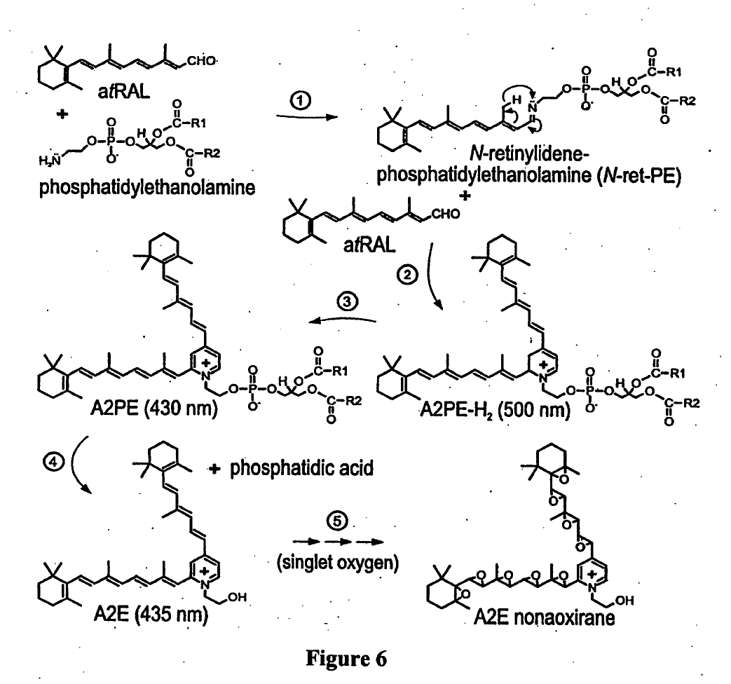

Biogenesis of A2E and A2E-oxiranes. A2E (dihydro-N-retinylidene-N-retinyl-ethanolamine), the major fluorophore of lipofuscin, is formed in macular or retinal degeneration, including Stargardt's macular degeneration, due to excess production of the visual-cycle retinoid, all-trans-retinaldehyde, a precursor of A2E; FIG. 6. Described herein are methods and devices for the diagnosis of ophthalmic diseases in patients by measuring the presence of fluorescent compounds, including compounds that result from excess production of all-trans-retinaldehyde and which can lead to further deterioration of the health of the eye. These compounds include N-retinylidene-phosphatidylethanolamine, dihydro-N-retinylidene-N-retinyl-phosphatidylethanolamine, N-retinylidene-N-retinyl-phosphatidylethanolamine, or dihydro-N-retinylidene-N-retinyl-ethanolamine, FIG. 6. The presence of N-retinylidene-phosphatidylethanolamine, dihydro-N-retinylidene-N-retinyl-phosphatidylethanolamine, or N-retinylidene-N-retinyl-phosphatidylethanolamine leads to production of A2E (dihydro-N-retinylidene-N-retinyl-ethanolamine) and A2E oxiranes.

Macular or Retinal Degeneration As discussed above, macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type.

In “dry” macular degeneration, the deterioration of the retina is associated with the formation of small yellow deposits, known as drusen, under the macula. This phenomena leads to a thinning and drying out of the macula. The location and amount of thinning in the retinal caused by the drusen directly correlates to the amount of central vision loss. Degeneration of the pigmented layer of the retina and photoreceptors overlying drusen become atrophic and cause a slow of central vision. This often occurs over a decade or more.

Most people who lose vision from age related macular degeneration have “wet” macular degeneration. In “wet” (neovascular) macular degeneration, abnormal blood vessels from the choroidal layer of the eye, known as subretinal neovascularization grow under the retina and macula. These blood vessels tend to proliferate with fibrous tissue, and bleed and leak fluid under the macula, causing the macula to bulge or move and distort the central vision. Acute vision loss occurs as transudate or hemorrhage accumulates in and beneath the retina. Permanent vision loss occurs as the outer retina becomes atrophic or replaced by fibrous tissues.

Stargardt Disease. Stargardt Disease is a macular dystrophy that manifests as a recessive form of macular degeneration with an onset during childhood. See e.g., Allikmets et al., Science, 277:1805-07 (1997). Stargardt Disease is characterized clinically by progressive loss of central vision and progressive atrophy of the RPE overlying the macula. Mutations in the human ABCA4 gene for RmP are responsible for Stargardt Disease. Early in the disease course, patients show delayed dark adaptation but otherwise normal rod function. Histologically, Stargardt Disease is associated with deposition of lipofuscin pigment granules in RPE cells.

Besides Stargardt Disease, mutations in ABCA4 have been implicated in recessive retinitis pigmentosa, recessive cone-rod dystrophy, and non-exudative age-related macular degeneration (AMD), see e.g., Lewis et al., Am. J. Hum. Genet., 64:422-34 (1999), although the prevalence of ABCA4 mutations in AMD is still uncertain. See Allikmets, Am. J. Hum. Gen., 67:793-799 (2000). Similar to Stargardt Disease, these diseases are associated with delayed rod dark-adaptation. Lipofuscin deposition in RPE cells is also seen prominently in AMD, see Kliffen et al., Microsc. Res. Tech., 36:106-22 (1997) and some cases of retinitis pigmentosa.

An eye doctor examining a patient at this stage may note the presence of these drusen, even though most people have no symptoms. When drusen have been noted on examination, monitoring will be needed over time. Many people over the age of 60 will have some drusen.

Methods of Detection of Macular or Retinal Degeneration. Known methods of determining whether a patient has macular or retinal degeneration can be carried out at a doctor's office. A simple test with an Amsler grid may help assess whether a patient is experiencing areas of distorted or reduced vision, both common symptoms of macular degeneration.

Identification of abnormal blood vessels in the eye can be done with an angiogram. This identification can help determine which patients are candidates for the use of a candidate substance or other treatment method to hinder or prevent further vision loss. Angiograms can also be useful for follow-up of treatment as well as for future evaluation of any new vessel growth.

A fluorescein angiogram (fluorescein angiography, fluorescein angioscopy) is a technique for the visualization of choroidal and retinal circulation at the back of the eye. Fluorescein dye is injected intravenously followed by multiframe photography (angiography) or ophthalmoscopic evaluation (angioscopy). Fluorescein angiograms are used in the evaluation of a wide range of retinal and choroidal diseases through the analysis of leakage or possible damage to the blood vessels that feed the retina.

Similarly, angiograms using indocyanine green can be used for the visualization circulation at the back of the eye. Wherein fluorescein is more efficient for studying retinal circulation, indocyanine is better for observing the deeper choroidal blood vessel layer. The use of indocyanine angiography is helpful when neovascularization may not be observed with fluorescein dye alone.

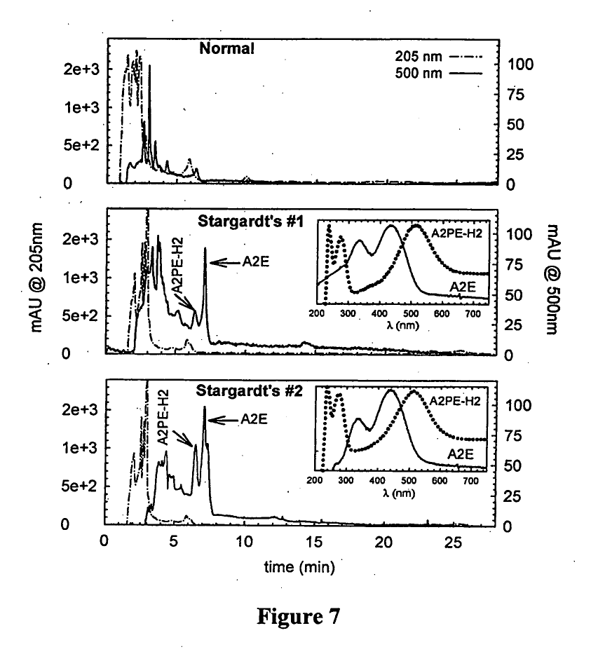

Fluorescent Detection of A2E and Intermediates. Each of the intermediates (N-retinylidene-phosphatidylethanolamine, dihydro-N-retinylidene-N-retinyl-phosphatidylethanolamine, and N-retinylidene-N-retinyl-phosphatidylethanolamine) in the A2E (dihydro-N-retinylidene-N-retinyl-ethanolamine) cascade contribute to fluorescence in lipofuscin, FIG. 6. Through the use of ABCA4 knock-out mice, the present invention has identified specific assays to detect these intermediates. Non-invasive methods for detecting and measuring the components of this cascade have been developed, at least in part, using ABCA4 knock-out mice. Early detection of A2E precursors in retinas extracted from mice was performed by impacting dissected eyes, in particular the rod outer segment discs, with an excitation light source and measuring the absorption of excess energy given off as light at different wavelengths, FIG. 7. This method also provides a diagnostic for effectiveness of a particular treatment, therapy, drug treatment and the duration of that effectiveness.

Eye samples are sliced in layers and prepared by known methods to those skilled in the art. A confocal scanning laser ophthalmoscope is used in order to detect the various layers of the eye sample. In particular, the retinal pigmented epithelium (RPE) and the Bruch's membrane are assayed. Those skilled in the art can choose from a variety of different excitation sources which may include an arc lamp or laser and use of various lights which include visible and infrared. Stimulation of the sample within the wavelength between 210 and 450 nm, 300 and 550 nm, and 220 and 460 nm, are preferred for detection of N-retinylidene-phosphatidylethanolamine, dihydro-N-retinylidene-N-retinyl-phosphatidylethanolamine, N-retinylidene-N-retinyl-phosphatidylethanolamine, and dihydro-N-retinylidene-N-retinyl-ethanolamine. After an excitation source encounters the sample, the autofluorescence from the sample is passed through a beam splitter which enables the accumulation of different emitted wavelengths. In order to detect significant wavelengths, the reflected emissions from the specimen are passed through several filters then enter a detector. Filters are essential for detecting autofluorescence. Different types of detectors are known to those skilled in the art and may include a CCD camera, photodiodes, photomultipliers, and video cameras, and the like, which may optimally receive emissions at selected wavelengths.

Unless defined otherwise, all technical and scientific terms used herein have the same meaning as is commonly understood by one of skill in the art to which this invention belongs. All patents and publications referred to herein are incorporated by reference.

As used herein, the term “ABCA4 gene” refers to a gene encoding the rim protein or RmP. The ABCA4 gene is also known as the ABCR gene.

As used herein, the term “anti-oxidant” refers to a synthetic or natural substance that can prevent, delay or otherwise inhibit the oxidation of a compound or biological substance.

As used herein, the term “camera” refers to a device for optically recording radiation.

As used herein, the term “deconvoluting” refers to the process of converting data, information and/or images into (at least in part) constituent components. For example, a fluorescence or absorbance spectrum that features a complex wave form can be mathematically deconvoluted into the separate absorbance or fluorescence peaks that comprise the complex wave form. Suitable mathematical procedures and algorithms are well-known in the art, and suitable software packages for deconvoluting data, information and/or images are commercially available.

As used herein, the term “dihydro-N-retinylidene-N-retinyl-ethanolamine” (also known as A2E) refers to a compound having the structure:

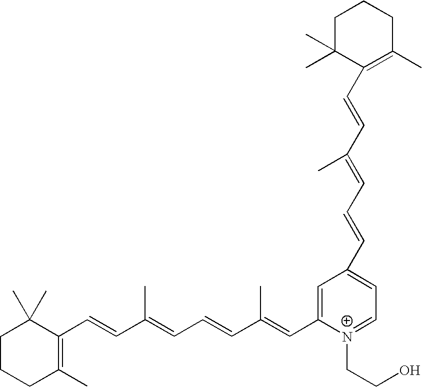

As used herein, the term “dihydro-N-retinylidene-N-retinyl-phosphatidylethanolamine” (A2PE-H₂) refers to a compound having the structure:

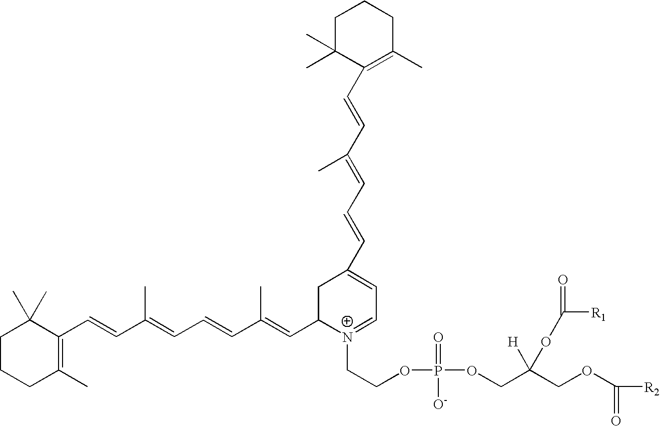

As used herein, the term “disruption of the visual cycle” or the like refers to any means for modulating the activity, directly or indirectly, of at least one enzyme involved in the visual cycle.

As used herein, the term “dispersing” refers to suspending a substance in another medium. Dispersing can include steps for homogenizing, fractionating, breaking up, fluidizing or decreasing the size of a substance in order to facilitate the suspending step.

As used herein, the term “drusen” refers to ophthalmoscopically visible, yellow-white hyaline excrescences of Bruch's membrane. They are deposits of cellular debris or collections of undigested waste material that can form under the retinal pigment epithelial cells. Accumulation of drusen and lipofuscin in Bruch's membrane may interfere with the transport of oxygen and nutrients to the retinal tissues, which ultimately leads to retinal pigment epithelial cell and photoreceptor dysfunction. In some families, drusen are heritable in an autosomal dominant fashion.

As used herein, the term “genetic testing” refers to a method for identifying those afflicted with hereditary diseases or conditions, and carriers of recessive disorders by means of DNA analysis.

As used herein, the term “magnification” refers to the amplification of an image.

As used herein, the term “mammal” refers to all mammals including humans. Mammals include, by way of example only, humans, non-human primates, cows, dogs, cats, goats, sheep pigs, rats, mice and rabbits.

As used herein, the term “measuring the emission fluorescence” refers to any means for either (a) detecting the presence of a fluorescent compound by detecting the presence of its fluorescence following excitation by some form of illumination, (b) measuring the amount of a fluorescent compound by measuring the intensity (absolute or relative) of the fluorescence emitted by the fluorescent compounds in a sample following excitation by some form of illumination, and (c) a combination of the above.

As used herein, the term “N-retinylidene-phosphatidylethanolamine” (also known as N-ret-PE) refers to a compound having the structure:

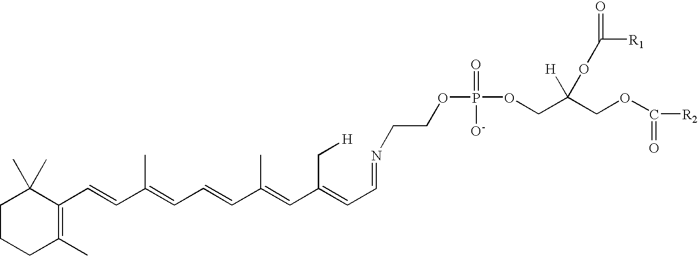

As used herein, the term “N-retinylidene-N-retinyl-phosphatidylethanolamine” (A2PE) refers to a compound having the structure:

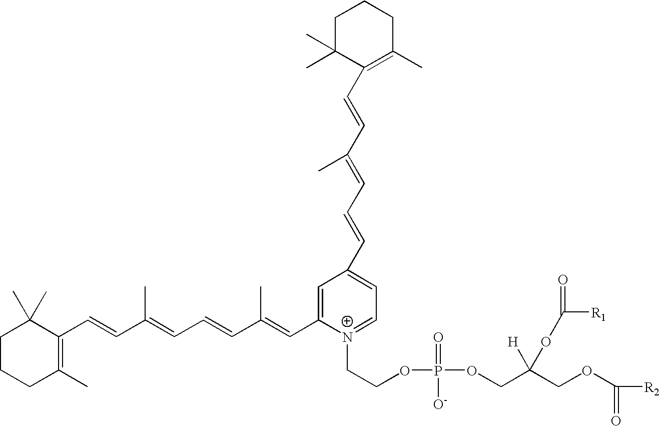

As used herein, the term “ophthalmic disease or condition” refers to any disease or condition involving the eye or related tissues. Non-limiting examples include diseases or conditions involving degeneration of the retina and/or macula, including the retinal and/or macular dystrophies and the retinal and/or macular degenerations.

As used herein, a retinyl derivative refers to a compound that can be produced by reacting one of the various cis or trans retinal isomers with another compound or series of compounds.

As used herein, 13-cis-retinyl derivative refers to a compound having the structure: 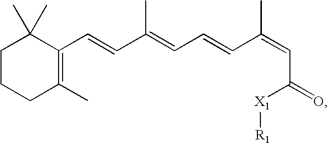
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoralkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof; and an effective amount of a second agent comprising an agent selected from the group consisting of an antioxidant, a mineral, an inducer of nitric oxide production, an anti-inflammatory agent, and a negatively charged phospholipid. These compounds are further described in U.S. Provisional Patent No. 60/602,675 herein incorporated by reference.

As used herein, all-trans-retinyl derivative refers to a compound having the structure: 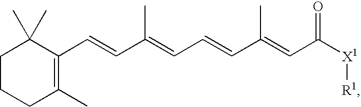
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoralkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof. These compounds are further described in U.S. Provisional Patent No. 60/582,293 herein incorporated by reference.

As used herein, the term “risk” refers to the probability that an event will occur.

As used herein, the term “spatial determination” refers to an image in which the absorbance and/or fluorescence of a sample is resolved spatially in x, y and/or z components. Such resolution may be in the form of pixels or other form of information: in addition to spatial information, such a unit of information may also contain data on the intensity and/or wavelength of light provided to, absorbed, and/or emitted from that region of the sample.

As used herein, the term “surrogate marker” refers to a laboratory measurement of biological activity within the body that indicates the effect of treatment or other stimulus on disease state.

As used herein, the term “whole eye illumination” refers to a method of providing light to an eye so as to illuminate at least a majority of the eye.

Treatment Methods, Dosages and Combination Therapies

There is a wide variety of treatments and therapies patients may consider for macular or retinal degenerations and dystrophies, which include: photodynamic therapy (PDT), low dose radiation therapy, submacular surgery, RPE transplantation, macular translocation surgery, laser treatment of drusen, and medications which can include an effective amount of a retinyl derivative, including derivatives of all-trans-retinal and 13-cis-retinal.

Other methods of treatment or therapies not mentioned herewith may also be used to treat macular or retinal degeneration. For examples of other treatments or therapies, see U.S. Provisional Application Ser. No. 60/582,293 filed Jun. 23, 2004; U.S. Provisional Application Ser. No. 60/602,675 filed on Aug. 18, 2004; U.S. Provisional Application Ser. No. 60/622,213 filed Oct. 25, 2004, 60/629,695, filed on Nov. 19, 2004, U.S. Provisional Application Ser. No. 60/660,904, filed on Mar. 11, 2005, U.S. Provisional Application Ser. No. 60/672,405, filed on Apr. 18, 2005; U.S. Non-Provisional patent application Ser. No. 11/150,641 filed Jun. 10, 2005; and PCT Patent Application No. US 2005/29455 filed Aug. 17, 2005, all of which have been herein incorporated by reference in their entirety.

Further combinations that may be used to provide benefit to an individual include the use of genetic testing to determine whether that individual is a carrier of a mutant gene that is known to be correlated with certain ophthalmic conditions. By way of example only, defects in the human ABCA4 gene are thought to be associated with five distinct retinal phenotypes including Stargardt disease, cone-rod dystrophy, age-related macular degeneration and retinitis pigmentosa. Such patients would be expected to find therapeutic and/or prophylactic benefit in the methods described herein.

### EXAMPLES

ABCA4 Knockout Mice. ABCA4 encodes rim protein (RmP), an ATP-binding cassette (ABC) transporter in the outer-segment discs of rod and cone photoreceptors. The transported substrate for RmP is unknown. Mice generated with a knockout mutation in the ABCA4 gene, are useful for the study of RmP function as well as for an in vivo screening of the effectiveness for candidate substances. These animals manifest the complex ocular phenotype: (i) slow photoreceptor degeneration, (ii) delayed recovery of rod sensitivity following light exposure, (iii) elevated atRAL and reduced atROL in photoreceptor outer-segments following a photobleach, (iv) constitutively elevated phosphatidylethanolamine (PE) in outer-segments, and (v) accumulation of lipofuscin in RPE cells.

Rates of photoreceptor degeneration can be monitored in treated and untreated wild-type and ABCA4−/− mice by two techniques. One is the study of mice at different times by ERG analysis and is adopted from a clinical diagnostic procedure. See Weng et al., Cell, 98:13-23 (1999). An electrode is placed on the comeal surface of an anesthetized mouse and the electrical response to a light flash is recorded from the retina. Amplitude of the α-wave, which results from light-induced hyperpolarization of photoreceptors, is a sensitive indicator of photoreceptor degeneration. ERGs are done on live animals. The same mouse can therefore be analyzed repeatedly during a time-course study. The definitive technique for quantitating photoreceptor degeneration is histological analysis of retinal sections. The number of photoreceptors remaining in the retina at each time point will be determined by counting the rows of photoreceptor nuclei in the outer nuclear layer.

Human Patients. For pre-testing, all human patients undergo a routine ophthalmologic examination including fluorescein angiography, measurement of visual acuity, electrophysiologic parameters and biochemical and rheologic parameters. Inclusion criteria are as follows: visual acuity between 20/160 and 20/32 in at least one eye and signs of ARMD such as drusen, areolar atrophy, pigment clumping, pigment epithelium detachment, or subretinal neovascularization. Patients with any of the following are excluded from the study: dementia; severe cardiac disease; history of malignancy or infection with hepatitis, or Treponema pallidum; and suitability for laser coagulation according to the guidelines of the Macular Photocoagulation Study Group.

### Example 1

### Fluorescence Analysis of A2E and A2PE-H₂ in Tissue Extracts

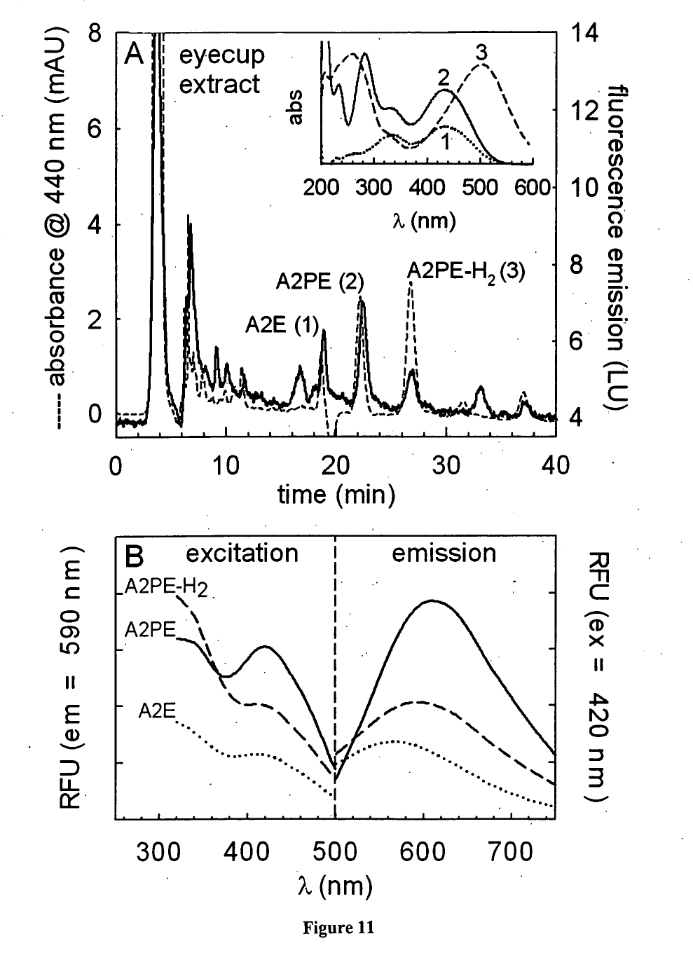

A. Preparing the Tissue Extract. Eyes are enucleated from euthanized mice and hemisected to reveal retina and retinal pigment epithelium (RPE). Retina is removed cleanly from underlying RPE with dissecting forceps. RPE is brushed from the underlying scleral tissue into 100-200 μl of PBS, pH 7.2 using a #2 camel hair brush. RPE cells are aspirated from the eyecup using a micro-pipette. Human post-mortem tissue is processed in a similar fashion. Tissues are homogenized by hand using a Duall glass-glass homogenizer following the addition of 500 μl chloroform/methanol (2:1, v/v). Samples are transferred to a borosilicate tube and lipids are extracted into 1 ml of chloroform. The organic extract is washed with 1 ml PBS, pH 7.2 and the samples are centrifuged at 3,000×g, 10 min. The chloroform phase is decanted and the aqueous phase is re-extracted with another 1 ml of chloroform. Following centrifugation, the chloroform phases are combined and the samples are taken to dryness under nitrogen gas. Sample residues are resuspended in 200-500 μl methanol and analyzed by HPLC (FIG. 11A).

B. Fluorescence Analysis of A2E and A2PE-H₂. Excitation spectra for A2E, A2PE, and A2PE-H2 are obtained in the range of 250-500 nm using an emission wavelength of 590 nm. Emission spectra for A2E, A2PE, and A2PE-H₂ are obtained in the range of 500-750 nm using an excitation wavelength of 420 nm. Bandpass filters (slit widths) are adjusted according to sample concentration. Data are obtained using a Jobin-Yvon Fluorolog 3 spectrofluorometer. Data are analyzed using Data Max software version 2.2 (FIG. 11B).

### Example 2

### Fluorescence Analysis of A2PE-H₂ in Whole Retina Explant

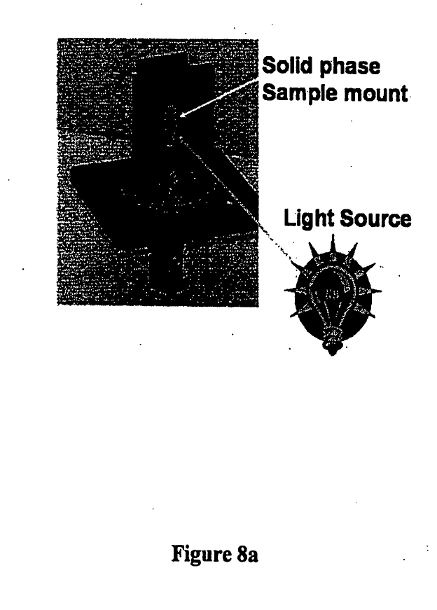

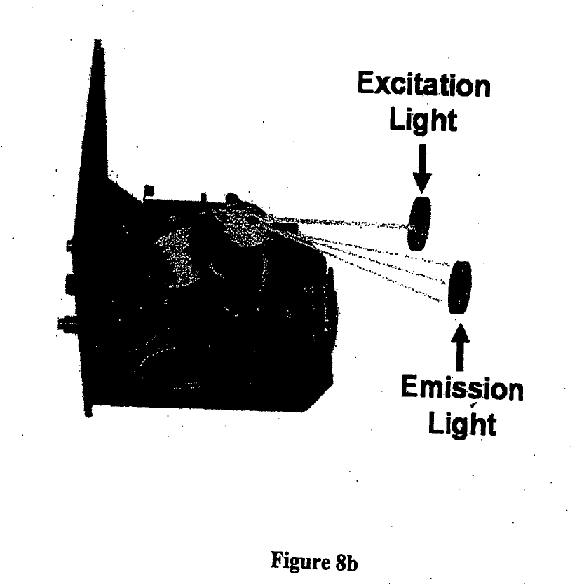

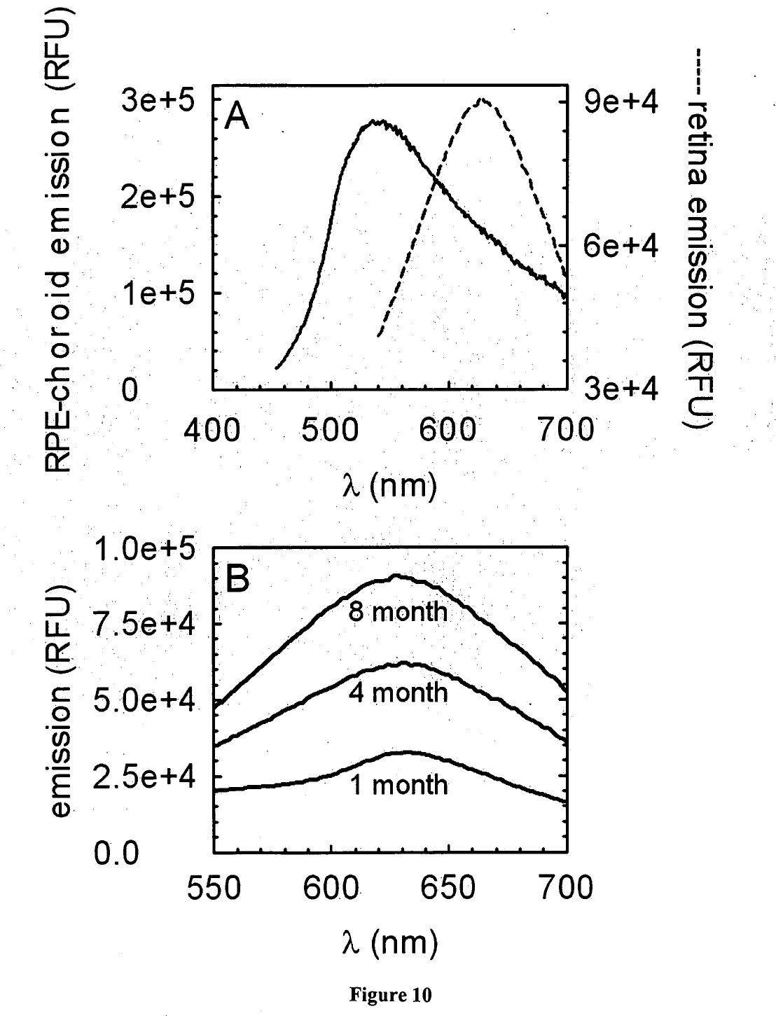

A. Preparing the Whole Retina and Retinal Epithelium Explants. Eyes are enucleated from euthanized mice and hemisected to reveal retina and retinal pigment epithelium (RPE). Retina is removed cleanly from underlying RPE with dissecting forceps. The remaining RPE/sclera are saved and stored separately. Samples of post-mortem human retina tissue are obtained as described above. The retina and RPE/sclera samples are moistened with PBS, pH 7.2 and placed separately into a solid phase sample mount so that the sample is oriented perpendicular to the incoming light (FIG. 8A). Emission spectra are obtained from the samples as described (FIG. 10A).

B. Fluorescence Analysis of A2PE-H₂. Front face fluorescence emission from retina samples are acquired at 22.5° relative to the incoming light. Excitation light is set to 480 nm and emission spectra are acquired from 500 nm to 650 nm using a Jobin-Yvon Fluorolog 3 spectrofluorometer. Bandpass filters (slit widths) are adjusted to optimize the fluorescence signal and minimize background. Data are analyzed using Data Max software version 2.2.

### Example 3

### Fluorescence Analysis of A2E and A2PE-H₂ in the Intact Eye of a Live Animal

A. Preparing the Intact Eye of a Live Animal. Live mice are treated with a mydriatic (e.g., atropine) in order to dilate the pupil. The mice are anesthetized and placed onto a modified sample cell carriage such that the right or left eye is oriented toward the incoming light. See FIG. 8b.

B. Fluorescence Analysis of A2E and A2PE-H₂ in the Intact Eye of a Live Animal. Front face fluorescence emission from intact eyes are acquired at 22.5° relative to the incoming light. Three analyses are performed: 1) Excitation light is set to 450 nm and emission spectra are acquired from 460 nm to 650 nm; 2) Excitation light is set to 480 nm and emission spectra are acquired from 490 nm to 650 nm; 3) Excitation light is set to 500 nm and emission spectra are acquired from 510 nm to 700 nm;

All data are acquired using a Jobin-Yvon Fluorolog 3 spectrofluorometer. Bandpass filters (slit widths) are adjusted to optimize the fluorescence signal and minimize background. Data are analyzed using Data Max software version 2.2.

### Example 4

### Comparison of Fluorescence Analysis of A2E and A2PE-H₂ in abcr−/− and Wild Type Mice

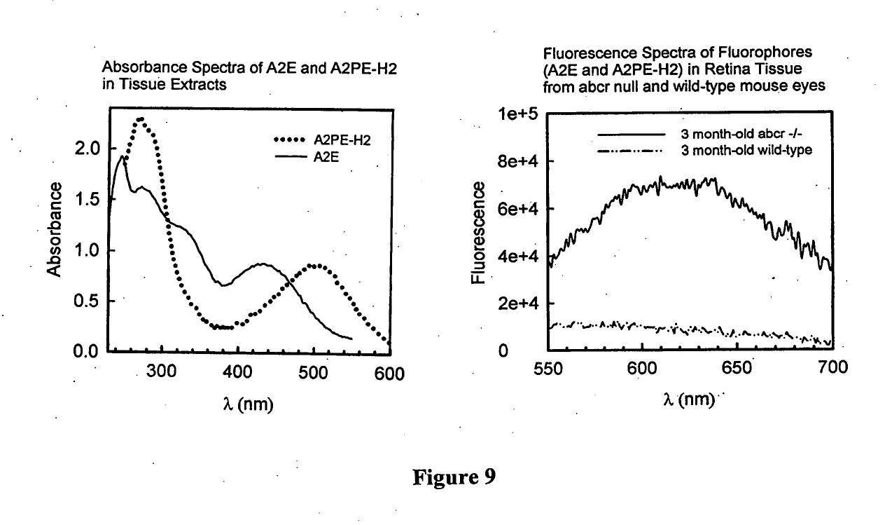

Absorbance spectra were obtained from analysis of tissue extracts by HPLC. Lipid soluble components were extracted from eyecups of abcr−/− null mutant mice. Fluorescence spectra were obtained by dissociating retina-RPE tissue with mild protease (dispase) in solution then scanning the entire sample. Comparison spectra are from age and strain matched wild type mice. See FIG. 9.

### Example 5

### Detecting the Presence of A2E and/or Precursors in a Sample Comprising the Eye of a Human Patient

A. Preparing the Intact Eye of a Human Patient. The eye of the human patient is treated to dilate the pupil. The patient's head is optionally secured to prevent movement. Optionally, patients can be anesthetized.

B. Fluorescence Analysis of A2E and A2PE-H₂ in the Intact Eye of a Human Patient. Front face fluorescence emission from intact eyes are acquired at 22.5° relative to the incoming light. Three analyses are performed: 1) Excitation light is set to 450 nm and emission spectra are acquired from 460 nm to 650 nm; 2) Excitation light is set to 480 nm and emission spectra are acquired from 490 nm to 650 nm; 3) Excitation light is set to 500 nm and emission spectra are acquired from 510 nm to 700 nm;

All data are acquired using a Jobin-Yvon Fluorolog 3 spectrofluorometer. Bandpass filters (slit widths) are adjusted to optimize the fluorescence signal and minimize background. Data are analyzed using Data Max software version 2.2.

### Example 6

### Monitoring the Effectiveness of Ophthalmic Treatment, Therapies or Drugs

Assessing the effectiveness of treatments, therapies or drugs which have an effect on macular or retinal degenerations and dystrophies can be a three step process which involves 1) taking the initial measurements of A2E and A2PE-H₂ in a subject, 2) providing treatment, therapy or drug to the subject, 3) taking measurements of A2E and A2PE-H₂ after step (2), and assessing results which would indicate that the treatment, therapy or drug may have a desired effect. A desired result may include a decrease or suspension in the amount of A2E and/or A2PE-H₂ accumulation in the eye(s) of the subject. Reiteration of steps 2-3 may be administered with or without intervals of non-treatment. Subjects may include but are not limited to mice and/or rats and/or human patients. Drug treatments may include but are not limited to (a) an all-trans-retinyl derivative refers to a compound having the structure: 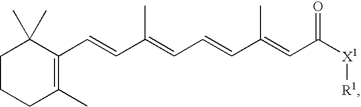
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoralkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof; (b) an effective amount of a second agent in addition to an effective amount of an all-trans-retinyl derivative, wherein the second agent is selected from the group consisting of an antioxidant, a mineral, an inducer of nitric oxide production, an anti-inflammatory agent, and a negatively charged phospholipid, or (c) a combination of 1.0 mg/kg per day isotretinoin with 600 mg vitamin C, 450 mg vitamin E, 30,000 IU vitamin A, 90 mg zinc and 2.5 mg copper and/or with an effective amount of an additional agent comprising an agent selected from the group consisting of an antioxidant, a mineral, an inducer of nitric oxide production, an anti-inflammatory agent, and a negatively charged phospholipid. Dosage of drug treatment for mice and/or rat and/or human subjects should be calculated based on weight. The all-trans-retinyl derivative can be selected from compounds in which (a) X¹ is NR², wherein R² is H or (C₁-C₄)alkyl; (b) wherein x is 0; (c) x is 1 and L¹ is —C(O)—; (d) R³ is an optionally substituted aryl; (e) R³ is an optionally substituted heteroaryl; (f) X¹ is NH and R³ is an optionally substituted aryl, including yet further embodiments in which (i) the aryl group has one substituent, (ii) the aryl group has one substituent selected from the group consisting of halogen, OH, O(C₁-C₄)alkyl, NH(C₁-C₄)alkyl, O(C₁-C₄)fluoroalkyl, and N[(C₁-C₄)alkyl]₂, (iii) the aryl group has one substituent, which is OH, (v) the aryl is a phenyl, or (vi) the aryl is naphthyl; (g) the compound is 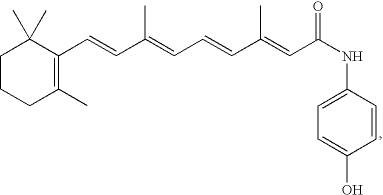
or an active metabolite, or a pharmaceutically acceptable prodrug or solvate thereof; (h) the compound is 4-hydroxyphenylretinamide, or a metabolite, or a pharmaceutically acceptable prodrug or solvate thereof; (i) the compound is 4-methoxyphenylretinamide, or (j) 4-oxo fenretinide, or a metabolite, or a pharmaceutically acceptable prodrug or solvate thereof.

All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method for measuring the presence of N-retinylidene-N-retinyl-phosphatidylethanolamine in a sample comprising illuminating the sample with source light having a wavelength between 300 and 550 nm, and measuring the emission fluorescence in the received light from the sample between 570 and 700 nm.

2. A method for measuring the presence of N-retinylidene-N-retinyl-ethanolamine in a sample comprising illuminating the sample with source light having a wavelength between 210 and 450 nm, and measuring the emission fluorescence in the received light from the sample between 470 and 700 nm.

3. The method of any of claims 1-2 wherein the sample comprises an eye of a human.

4. The method of any of claims 1-2 wherein the measuring step comprises use of confocal scanning ophthalmoscopy.

5. The method of any of claims 1-2 wherein the emission fluorescence is used to diagnose an ophthalmic disease or condition.

6. The method of claim 5 wherein the ophthalmic disease or condition is Stargardt Disease.

7. The method of claim 5 wherein the ophthalmic disease or condition is dry form age-related macular degeneration.

8. The method of claim 5 wherein the ophthalmic disease or condition is wet form age-related macular degeneration.

9. The method of any of claims 1-2 wherein the emission fluorescence is used to monitor the formation of drusen in an eye of a mammal.

10. The method of any of claims 1-2 wherein the emission fluorescence is used to monitor the formation of lipofuscin in an eye of a mammal.

11. The method of any of claims 1-2 wherein the emission fluorescence is used as a risk factor for an ophthalmic disease or condition in an eye of a mammal.

12. The method of any of claims 1-2 wherein the emission fluorescence is used as a surrogate marker for an ophthalmic disease or condition in an eye of a mammal.

13. The method of any of claims 1-2 wherein the emission fluorescence is used to monitor the progress of an ophthalmic disease or condition in an eye of a mammal.

14. The method of any of claims 1-2 wherein the emission fluorescence is used to determine whether an eye of a mammal should receive treatment for an ophthalmic disease or condition.

15. The method of any of claims 1-2 wherein the emission fluorescence is used to measure the effectiveness of a treatment.

16. The method of claim 15 wherein the treatment comprises administering to the mammal at least once an effective amount of a first compound having the structure: 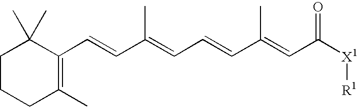
wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; or an active metabolite, or a pharmaceutically acceptable prodrug or solvate thereof; provided that R is not H when both x is 0 and L¹ is a single bond.

17. The method of any of claims 16, wherein x is 0.

18. The method of any of claims 16, wherein X¹ is NH and R³ is phenyl group, wherein the phenyl group has one substituent.

19. The method of claim 18, wherein the substituent is a moiety selected from the group consisting of halogen, OH, O(C₁-C₄)alkyl, NH(C₁-C₄)alkyl, O(C₁-C₄)fluoroalkyl, and N[(C₁-C₄)alkyl]₂.

20. The method of claim 19, wherein the substituent is OH or OCH₃.

21. The method of any of claims 16, wherein the compound is 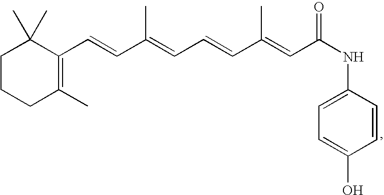
or an active metabolite, or a pharmaceutically acceptable prodrug or solvate thereof.
